SEQUENCE LISTING (1 sequence)

SEQ ID NO: 1 - DNA, 1194 bp, Rotavirus A
  source 1..1194: mol_type = genomic DNA
atggaggttctgtactcactgtcacaaactttgaaagatgctagagataagattgttgagggtacattgtattcaaatgtaagtgacttaattcaacaatttaatcaaatgatagttactatgaatgggaatgattttcaaacaggaggaataggaaatttgccaattaggagttggacttttgacttcggattacttggtacaactttacttaacctagatgcaaattatgttgaaaatgcaagaactaccattgaatatttcattgattttatagataatgtgtgtatggatgaaatagctagagaatcacaacgaaatggaatagctccacaatctgaagcactaagaaagctgtcaggtattagatttaaaagaatcaattttgataattcatctgattacattgaaaattggaatttgcaaaataggcgacagcgtactggatttgtattccacaaaccaaacatacttccatattcggcgtcattcactctaaatagatcacagccagcacatgataatttgatgggaaccatgtggattaatgctggatcagagattcaggtagctggatttgattactcgtgcgcttttaatgcaccagcaaatattcaacagtttgaacatgtcgtaccattaagacgcgctcttacaacagctacaattactttgttgccagatgctgaaagattcagttttccgagagttatcaattcagctgatggtactactacatggtatttcaatccagttatttttagaccaagtaatgtagaagttgaatttctgctaaatggacaaataattaacacgtaccaagcacggtttggaactattgtggctaggaactttgatactattcgtttgtcatttcaattagtacgaccaccgaatatgacaccagcagttgcaaatctgtttccgcaagcaccaccatttttatttcatgctacagccggacttacattacggattgaatctgcaatttgtgaatcagtgcttgcggatgcttctgaaaccttgttggcaaatgtaacatcagttcgtcaggaatatgctataccagttggaccagtattcccaccaggtatgaattggaccgagttgattacaaactattcaccttcgagagaagataatctacaacgtattttcacagtagcctccattagaagcatgctgattaagtga

(19) United States
(12) Patent Application Publication
(10) Pub. No.: US 2024/0345087 A1
(43) Pub. Date: Oct. 17, 2024

(54) ELISA DETECTION KIT AND DETECTION METHOD FOR IGA ANTIBODY AGAINST PORCINE ROTAVIRUS GROUP A

(71) Applicants: Juye Xinhao Agriculture and Animal Husbandry Co. LTD, Shandong (CN); New Hope Liuhe Co., Ltd, Sichuan (CN); Xiajin New hope Liuhe agriculture and animal husbandry Co., LTD, Shandong (CN); Shandong new hope Liuhe Group Co., Ltd, Shandong (CN); Shandong New hope Liuhe Agriculture and Animal Husbandry Technology Co. LTD, Shandong (CN); New Hope Group Limited, Sichuan (CN)

(72) Inventors: Xiaowen Li, Shandong (CN); Jiapei Huang, Shandong (CN); Yang Li, Shandong (CN); Mengli QIAO, Shandong (CN); Jie Qing, Shandong (CN); Zewei WANG, Shandong (CN); Qiannan YU, Shandong (CN)

(73) Assignees: Juye Xinhao Agriculture and Animal Husbandry Co. LTD, Shandong (CN); New Hope Liuhe Co., Ltd, Sichuan (CN); Xiajin New hope Liuhe agriculture and animal husbandry Co., LTD, Shandong (CN); Shandong new hope Liuhe Group Co., Ltd, Shandong (CN); Shandong New hope Liuhe Agriculture and Animal Husbandry Technology Co. LTD, Shandong (CN); New Hope Group Limited, Sichuan (CN)

(21) Appl. No.: 18/633,527

(22) Filed: Apr. 12, 2024

(30) Foreign Application Priority Data
Apr. 17, 2023 (CN) 202310406671.7

(51) Int. Cl. G01N 33/569; G01N 33/543

(52) U.S. Cl. CPC G01N 33/56983; G01N 33/54386; G01N 2333/14; G01N 2469/20

(57) ABSTRACT

This invention provides an ELISA detection kit and method for IgA antibody against Porcine rotavirus group A, belonging to the field of animal husbandry detection technology. The ELISA detection method of this invention utilizes popular PoRV variant strains as parental strains to successfully express recombinant VP6 fusion protein in vitro. Subsequently, the VP6 fusion protein was used as an antigen to be coated onto enzyme-labeled plate to establish a novel ELISA detection method. The method provided by this invention possesses strong sensitivity, high specificity, robust stability, good reproducibility, and ease of preparation, and holds important clinical practical significance for the diagnosis, prevention and control of PoRV.

### CROSS-REFERENCE TO RELATED APPLICATION

This application claims the priority benefit of China application no. 202310406671.7, filed on Apr. 17, 2023. The entirety of each of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Apr. 10, 2024, is named 144883_SEQUENCELISTING and is 3,188 bytes in size.

### BACKGROUND OF THE INVENTION

### 1. Technical Field

This invention belongs to the field of animal husbandry detection technology, in particular relates to an ELISA detection kit and method for IgA antibody against Porcine rotavirus group A.

### 2. Background Art

Porcine rotavirus (PoRV) belongs to the family Reoviridae, in which Porcine rotavirus group A (RVAs) was one of the most important gut pathogens leading to severe dehydrating diarrhea in suckling piglets. The viral genome consists of 11 double-stranded RNA fragments encoding 6 structural proteins (VP1-VP4, VP6, VP7) and 6 non-structural proteins (NSP1-NSP6). The external envelope membrane capsid proteins VP4 and VP7 of rotavirus independently produce neutralizing antibodies, which can be classified into P-type and G-type depending on the two proteins. In recent years, the epidemic of porcine RVAs in China has become more and more widespread, especially the emergence of novel G and P combination variants. Therefore, detection of antibody levels after effective vaccine immunization and immunization in pigs is one of the key issues for effective prevention and control of PoRV, and it is crucial to establish a novel detection method based on antigenic proteins associated with novel variant strains as soon as possible.

VP6 was the main structural protein in rotavirus particles, and antibodies targeting the intermediate capsid protein VP6 protein play a key role in preventing rotavirus infection. At the same time, VP6 has high immunogenicity and antigenicity, and the current popular genotype of porcine rotavirus VP6 in China was group A, so recombinant VP6 based on group A is the most ideal target for the diagnostic detection of PoRV antibody, which was used to evaluate the ability to help prevent rotavirus disease in the context of porcine group infection and vaccination.

In addition, a large number of studies have shown that the presence of anti-rotavirus IgA antibodies rather than IgG antibodies in the gut and serum after rotavirus infection was highly correlated with the protective power produced, and was the most relevant factor to protect pigs from rotavirus disease. Piglets mainly obtained passive immune protection against porcine rotavirus by obtaining maternal IgA antibodies through sow milk, wherein the mucosal immunity of IgA antibodies in colostrum is the best way for piglets to obtain PoRV immune protection. As an important effector of the mucosal immune system, IgA has the main characteristics of multi-chain, mucosal affinity and resistance to proteases, which contributes to the affinity of antibodies to viruses, and has a variety of biological functions such as inhibiting adhesion, immune exclusion and neutralizing viruses. The level of IgA antibodies in the milk of sows after immunization with porcine rotavirus vaccine was an important indicator to evaluate the immunogenicity of vaccines. Therefore, the establishment of a safe, stable and easy-to-prepare IgA antibody detection method was of great clinical significance for the prevention and treatment of porcine rotavirus.

### SUMMARY OF THE INVENTION

In view of this, the object of this invention was to provide an ELISA detection method for VP6 fusion recombinant protein IgA antibody based on against porcine rotavirus group A, which satisfies the detection of serum and colostrum samples at the same time, and realizes the detection of PoRV immune protection effect in piglets at the same time.

In order to realize the above purpose, the invention provides the following technical scheme:

First, this invention provides an ELISA detection kit and detection method for IgA antibody against Porcine rotavirus group A, and said the detection kit comprises an enzyme-label plate coated with VP6 fusion recombinant protein, a sample diluent, a wash solution, a substrate chromogenic solution, and a stop solution.

Preferably, said the VP6 fusion recombinant protein being synthesized use against porcine rotavirus group A as a template.

Preferably, the encoding sequence of the VP6 fusion recombinant protein was shown in SEQ ID NO.1.

Preferably, the coating concentration of VP6 fusion recombinant protein was 3.5 μg/mL.

Preferably, the coating time of the VP6 fusion recombinant protein was 10-14 h.

Second, this invention provides an ELISA detection method for IgA antibody against Porcine rotavirus group A, and said the method comprises the following steps:
Coating: The VP6 fusion recombinant protein is diluted to 3.5 μg/mL using a 0.05 mol/L Na₂CO₃—NaHCO₃ buffer solution with a pH of 9.6 and 100 μL of the diluted protein solution was added to each well, and adsorbed at 4° C. for 14 h;
After coating, the liquid in the wells is discarded, and 250 μL of PBS wash solution containing 0.05% Tween-20 is added to each well. The wells were washed three times, with the final wash being tapped dry.
Blocking: 200 μL of PBS blocking solution containing 5% BSA and 0.05% Tween-20 is added to each well and blocked at 37° C. for 1 h;
After blocking, the liquid in the wells was discarded, 250 μL of wash solution was added to each well. The wells were washed three times, with the final wash being tapped dry;
Primary antibody incubation: dilute the milk sample to be tested with sample diluent at a ratio of 1:100 or dilute the serum sample to be tested with sample diluent at a ratio of 1:200, 100 L was added to each well and incubated at 37° C. for 60 min;
The liquid in the wells is discarded, 250 μL of wash solution is added to each well. The wells were washed five times, with the final wash being tapped dry;
Secondary antibody incubation: dilute the enzyme-labeled secondary antibody with antibody diluent at a ratio of 1:10000, 100 μL was added to each well and incubated at 37° C. for 60 min;
The liquid in the wells is discarded, 250 μL of wash solution is added to each well. The wells were washed five times, with the final wash being tapped dry;
Chromogenic time and reading: 100 μL of single-component TMB substrate chromogenic solution is added to each well, and the reaction was allowed to proceed in dark at 37° C. for 15 minutes. Subsequently, 100 μL of stop solution is added to each well, and the OD value at 450 nm was read using the enzyme-labeled instrument;
The described method was used for non-disease diagnostic purposes.

Preferably, said the VP6 fusion recombinant protein being synthesized us Porcine rotavirus group A as a template.

Preferably, said the encoding sequence of the VP6 fusion recombinant protein was shown in SEQ ID NO.1.

Preferably, said the cut-off value of the milk sample to be tested was 0.262;

When the OD value ≥0.262, the test result was positive, and when the OD value <0.262, the test result was negative.

Preferably, the cut-off value of the serum sample to be tested was 0.319;
When the OD value ≥0.319, the test result was positive, and when the OD value <0.319, the test result was negative.

The beneficial effects of this invention are that:

This invention provides an ELISA detection method for VP6 fusion recombinant protein IgA antibody based on against Porcine rotavirus group A. This invention takes the popular PoRV variant strains as the parental strains to successfully express the VP6 recombinant protein as the coating antigen in vitro, and successfully establishes a PoRV-specific ELISA detection method for IgA antibody with strong sensitivity, high specificity, stability, good repeatability and ease of preparation, which basically meets the detection of two sample types of porcine serum and porcine colostrum at the same time, and has important clinical practical significance for the clinical diagnosis and prevention and control of PoRV.

### BRIEF DESCRIPTION OF THE DRAWINGS

FIG. 1 is an electropherogram of the enzyme digestion product of the pColdII-VP6 recombinant expression vector constructed by this invention;

FIG. 2 is a result of the VP6 fusion recombinant protein purified by nickel column affinity chromatography.

FIG. 3 is the specific detection result of the detection method of this invention;

FIG. 4 is the sensitivity test result of the detection method of this invention.

### DETAILED DESCRIPTION OF THE EMBODIMENTS

A variety of exemplary embodiments of this invention were described in detail, and such detailed description shall not be regarded as a limitation of this invention, but shall be understood as a more detailed description of certain aspects, characteristics and embodiments of this invention.

It should be understood that the terms described in the invention were only intended to describe special embodiments and were not intended to limit the invention. In addition, with respect to the numerical range in this invention, it should be understood that each intermediate value between the upper and lower limits of the range was also specifically disclosed. The intermediate value in any stated value or range of representations, and each smaller range between any other stated value or intermediate value in said range was also included in this invention. The upper and lower limits of these smaller ranges can be included or excluded independently.

### Example 1: Construction of pColdII-VP6 Recombinant Expression Vector

(1) Referring to the VP6 sequence (SEQ ID NO.1) of the CHN/SCCD/A/2017 strain in GenBank, the sequence was synthesized by Sangon Biotech (Shanghai) Co., Ltd.
(2) After enzymatic digestion and purification, the synthetic product was inserted into the BamHI/HindIII multiple cloning site of the pColdII-VP6 vector. The expected result was verified through sequencing, thus construct the recombinant expression vector pColdII-VP6.

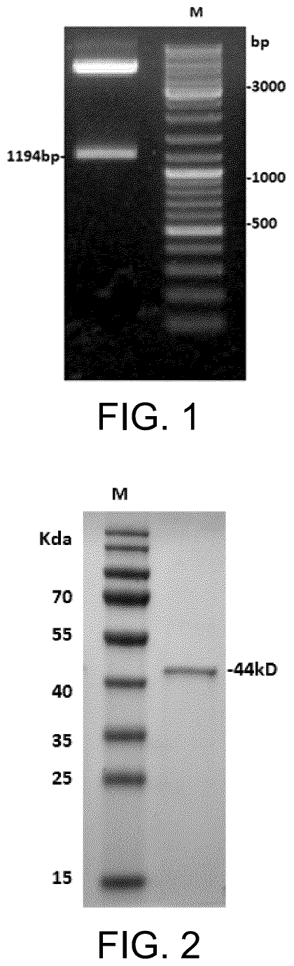

As shown in FIG. 1 of enzymatic digestion result, the target gene was correctly inserted into the pColdII-VP6 vector.

### Example 2: Expression and Purification of Recombinant Protein

(1) The constructed recombinant plasmid pColdII-VP6 was transformed into BL21 competent cells to obtain the recombinant expression strain BL21-pColdII-VP6.
(2) The cells were cultured at 37° C. with 220 rpm oscillation for 2-3 hours until the OD value at 600 nm reached 0.6-0.8. Subsequently, IPTG with a final concentration of 0.1 mmol/L was added, and the cells were induced for expression at 16° C. with 220 rpm oscillation culture.
(3) The bacteria were collected by centrifugation and disrupted and lysed by ultrasonic treatment.
(4) The cell lysate was centrifuged to separate the supernatant and precipitate. Both fractions were analyzed by SDS-PAGE to determine the expression form of the VP6 recombinant protein. Samples containing the target protein were collected, and the pColdII-VP6 recombinant protein was purified by nickel column affinity chromatography according to the Ni-NTA Superflow Cartridge manual. The purified protein samples were analyzed by SDS-PAGE.

As shown in FIG. 2, the soluble protein was purified using nickel column affinity chromatography, resulting in a purity of pColdII-VP6 recombinant protein above 95%.

### Example 3: Determination of Optimal Antigen Coating Concentration and Optimal Secondary Antibody Working Concentration

(1) A matrix assay was used to determine the optimal antigen coating concentration and optimal secondary antibody working concentration. The VP6 fusion recombinant protein was diluted to six concentrations by coating buffer: 7 μg/mL, 3.5 μg/mL, 1.75 μg/mL, 0.625 μg/mL, 0.3125 μg/mL and 0.156 μg/mL. Each concentration of the VP6 fusion recombinant protein was coated on one row of the ELISA plate, with 100 μL per well.
(2) The HRP-labeled 3 against porcine IgA antibody was sequentially diluted at ratios of 1:5000, 1:10000 and 1:20000 by antibody diluent. Two columns of wells were added for each dilution, with 100 μL per well, forming a matrix pattern.
(3) The antigen concentration and secondary antibody dilution that produced the highest ratio of positive milk OD value at 450 nm to negative milk OD value at 450 nm (P/N value) were selected as the optimal antigen coating concentration and optimal secondary antibody working concentration. The results are presented in Table 1.

TABLE 1
Determination of the optimal antigen coating concentration
and the optimal secondary antibody working concentration

| Antigen | Enzyme-labeled secondary antibody dilution | | | | | | | | |
|---|---|---|---|---|---|---|---|---|---|
| coating | 1:5000 | | | 1:10000 | | | 1:20000 | | |
| concentration | P | N | | P | N | | P | N | |
| (μg/mL) | OD_{450 nm} | OD_{450 nm} | P/N | OD_{450 nm} | OD_{450 nm} | P/N | OD_{450 nm} | OD_{450 nm} | P/N |
| 7.000 | 1.336 | 0.219 | 6.100 | 1.056 | 0.152 | 6.947 | 0.717 | 0.097 | 7.392 |
| 3.500 | 0.972 | 0.154 | 6.312 | 0.962 | 0.111 | 8.667 | 0.522 | 0.089 | 5.865 |
| 1.250 | 0.855 | 0.135 | 6.333 | 0.685 | 0.121 | 5.661 | 0.430 | 0.083 | 5.181 |
| 0.625 | 0.614 | 0.128 | 4.797 | 0.474 | 0.093 | 5.097 | 0.332 | 0.073 | 4.548 |
| 0.313 | 0.496 | 0.109 | 4.550 | 0.353 | 0.082 | 4.305 | 0.225 | 0.071 | 3.169 |
| 0.156 | 0.391 | 0.093 | 4.204 | 0.278 | 0.076 | 3.658 | 0.195 | 0.064 | 3.047 |

As shown in Table 1, the concentration of VP6 fusion recombinant protein was diluted to 1.25˜7 μg/mL, and the dilution of enzyme-labeled secondary antibody was in the range of 1:5000˜20000, among which the highest P/N value was obtained when the concentration of VP6 fusion recombinant protein was 3.5 μg/mL and the dilution of enzyme-labeled secondary antibody was 1:10000, indicating that the dose combination was the optimal combination.

### Example 4: Determination of Optimal Antigens Coating Conditions

The optimal antigen coating concentration and the optimal secondary antibody working concentration obtained in example 2 were used as test conditions. Incubation were set into three test groups at 37° C. for 1 h, 37° C. for 2 h and 4° C. overnight (15˜16 h) for ELISA detection, and the P/N value (P/N value=OD_{450 nm} mean value of positive samples/OD_{450 nm} mean value of negative samples) is calculated, selecting a maximum P/N value as an optimal antigen coating condition. The results are shown in Table 2.

TABLE 2
Determination of optimal antigen coating conditions

| | Antigen coating conditions | | |
|---|---|---|---|
| Groups | 37° C. 1 h | 37° C. 2 h | 4° C. overnight |
| Positive milk OD_{450 nm} | 0.732 | 0.824 | 0.965 |
| | 0.724 | 0.867 | 0.972 |
| | 0.735 | 0.888 | 0.983 |
| Positive milk OD_{450 nm} mean | 0.730 | 0.860 | 0.973 |
| value | | | |
| Positive milk OD_{450 nm} | 0.098 | 0.102 | 0.111 |
| | 0.093 | 0.107 | 0.112 |
| | 0.092 | 0.103 | 0.114 |
| Negative milk OD_{450 nm} mean | 0.094 | 0.104 | 0.112 |
| value | | | |
| P/N value | 7.742 | 8.266 | 8.665 |

As shown in Table 2, the P/N value of the overnight coating at 4° C. and the 1˜2 h coating at 37° C. have obvious advantages, so the method for the VP6 fusion recombinant protein coated with overnight coating at 4° C. was optimal.

### Example 5: Determination of Optimal Blocking Conditions

The Enzyme-labeled plate is coated with the optimal antigen coating concentration and the optimal coating conditions. Three test groups were set up for 37° C. blocked for 1 h, 37° C. blocked for 2 h and 4° C. overnight. The OD 450 nm value of positive and negative milk in each group is measured separately, and the P/N value is calculated to determine the optimal blocking time, and the results are shown in Table 3.

TABLE 3
Determination of optimal blocking conditions

| | Blocking conditions | | |
|---|---|---|---|
| Groups | 37° C. 1 h | 37° C. 2 h | 4° C. overnight |
| Positive milk OD_{450 nm} | 0.975 | 0.825 | 0.735 |
| | 0.982 | 0.863 | 0.721 |
| | 0.983 | 0.889 | 0.724 |
| Positive milk OD_{450 nm} mean | 0.980 | 0.859 | 0.727 |
| value | | | |
| Negative milk OD_{450 nm} | 0.113 | 0.101 | 0.096 |
| | 0.111 | 0.103 | 0.094 |
| | 0.114 | 0.105 | 0.097 |
| Negative milk OD_{450 nm} mean | 0.113 | 0.103 | 0.096 |
| value | | | |
| P/N value | 8.698 | 8.340 | 7.596 |

As shown in Table 3, the P/N value of the blocking solution at 37° C. for 1˜2 h has obvious advantages over at 4° C. overnight, and the enzyme-labeled plate of this invention was treated with the at 37° C. for 1 h blocking method.

### Example 6: Determination of Optimal Milk Dilution and Incubated Time

(1) ELISA test is carried out under the determined optimal conditions, and the male and negative milk are diluted at ratios of 1:50, 1:100, 1:200, and 1:400 times respectively, with 100 L/well.
(2) After adding the diluted milk, three test groups were setup, the first group is incubated at 37° C. for 30 min, the second group is incubated at 37° C. for 45 min, and the third group is incubated at 37° C. for 60 min.
(3) The OD 450 nm value of positive and negative milk in each group is measured separately, and the P/N value is calculated to determine the optimal milk dilution and incubated time, and the results are shown in Table 4.

TABLE 4
Optimization of milk dilution and incubated time

| | Milk incubated time | | | | | | | | |
|---|---|---|---|---|---|---|---|---|---|
| | 30 min | | | 45 min | | | 60 min | | |
| Milk | OD_{450 nm} mean | | | OD_{450 nm} mean | | | OD_{450 nm} mean | | |
| dilution | value | | | value | | | value | | |
| factor | P | N | P/N | P | N | P/N | P | N | P/N |
| 1:50 | 1.112 | 0.203 | 5.478 | 1.119 | 0.256 | 4.371 | 1.268 | 0.295 | 4.298 |
| 1:100 | 0.856 | 0.112 | 7.643 | 0.912 | 0.117 | 7.795 | 0.973 | 0.121 | 8.041 |
| 1:200 | 0.763 | 0.102 | 7.480 | 0.792 | 0.106 | 7.472 | 0.837 | 0.109 | 7.679 |
| 1:400 | 0.435 | 0.089 | 4.888 | 0.487 | 0.094 | 5.181 | 0.526 | 0.096 | 5.479 |

As shown in Table 4, the obtained P/N value has obvious advantages in the range of milk dilution of 1:50˜200, therefore, the milk samples of this invention was detected by a method with a dilution of 1:100 and an incubated time of 1 h.

### Example 7: Determination of Optimal Serum Dilution and Incubated Time

The ELISA test was performed under the determined optimal conditions, and the negative and negative serum are diluted at ratios of 1:50, 1:100, 1:200, and 1:400 respectively, with 100 μL/well. After adding the diluted serum, three test groups were setup, the first group is incubated at 37° C. for 30 min, the second group is incubated at 37° C. for 45 min, and the third group is incubated at 37° C. for 60 min. The positive and negative serum OD 450 nm values of each group are measured separately, and the P/N values are calculated to determine the optimal serum dilution and incubated time, and the results are shown in Table 5.

TABLE 5
Optimization of serum dilution and incubated time

| | Serum incubated time | | | | | | | | |
|---|---|---|---|---|---|---|---|---|---|
| | 30 min | | | 45 min | | | 60 min | | |
| Serum | OD_{450 nm} mean | | | OD_{450 nm} mean | | | OD_{450 nm} mean | | |
| dilution | value | | | value | | | value | | |
| factor | P | N | P/N | P | N | P/N | P | N | P/N |
| 1:50 | 1.728 | 0.375 | 4.608 | 1.862 | 0.395 | 4.714 | 1.879 | 0.398 | 4.721 |
| 1:100 | 1.546 | 0.311 | 4.971 | 1.632 | 0.327 | 4.991 | 1.657 | 0.324 | 5.114 |
| 1:200 | 1.347 | 0.258 | 5.221 | 1.457 | 0.269 | 5.416 | 1.465 | 0.268 | 5.466 |
| 1:400 | 0.766 | 0.198 | 3.869 | 0.943 | 0.211 | 4.469 | 0.965 | 0.214 | 4.509 |

As shown in Table 5 that in the range of serum dilution of 1:100˜400, the obtained P/N value has obvious advantages, therefore, the serum samples of this invention were detected by a method with a dilution of 1:200 and an incubated time of 1 h.

### Example 8: Determination of the Optimal Secondary Antibody Incubated Time

(1) Perform the ELISA test under the determined optimal conditions, and add enzyme-labeled secondary antibody (100 μL/well). Three test groups are set up, the first group is incubated at 37° C. for 30 min, the second group is incubated at 37° C. for 45 min, and the third group is incubated at 37° C. for 60 min.
(2) The OD450 nm value of the positive and negative milk in each group is measured, and the P/N value is calculated to determine the optimal incubated time of the secondary antibody, and the results are shown in Table 6.

TABLE 6
Determination of optimal secondary antibody incubated time

Enzyme-labeled secondary
antibody incubated time

| | 37° C. 60 | 37° C. 45 | 37° C. 30 |
|---|---|---|---|
| Groups | min | min | min |
| Positive milk OD_{450 nm} | 0.972 | 0.825 | 0.735 |
| | 0.984 | 0.863 | 0.721 |
| | 0.983 | 0.889 | 0.724 |
| Positive milk OD_{450 nm} | 0.980 | 0.859 | 0.727 |
| mean value | | | |
| Negative milk OD_{450 nm} | 0.110 | 0.105 | 0.095 |
| | 0.113 | 0.102 | 0.098 |
| | 0.111 | 0.104 | 0.097 |
| Negative milk OD_{450 nm} | 0.111 | 0.104 | 0.097 |
| mean value | | | |
| P/N value | 8.799 | 8.286 | 7.517 |

As shown in Table 6, a secondary antibody incubation solution at 37° C. for 60 minutes was optimal.

### Example 9: Determination of the Optimal Substrate Chromogenic Time

(1) The ELISA test is carried out under the determined optimal conditions, and three test groups are set up after adding TMB (with 100 μL/well), the first group was incubated at room temperature for 10 min, the second group was incubated at room temperature for 15 min, and the third group was incubated at room temperature for 20 min;
(2) After the chromogenic process was completed, 100 μL of stop solution is added to each well to terminate the chronography, the OD 450 nm value was read, and the P/N value is calculated to determine the optimal substrate chromogenic time, and the results are shown in Table 7.

TABLE 7
Determination of TMB chromogenic time

TMB chromogenic time

| | 37° C. 10 | 37° C. 15 | 37° C. 20 |
|---|---|---|---|
| Groups | min | min | min |
| Positive milk OD_{450 nm} | 0.737 | 0.972 | 1.112 |
| | 0.728 | 0.982 | 1.106 |
| | 0.724 | 0.988 | 1.103 |
| Positive milk OD_{450 nm} | 0.730 | 0.981 | 1.107 |
| mean value | | | |
| Negative milk OD_{450 nm} | 0.097 | 0.110 | 0.212 |
| | 0.098 | 0.113 | 0.214 |
| | 0.097 | 0.111 | 0.119 |
| Negative milk OD_{450 nm} | 0.097 | 0.111 | 0.182 |
| mean value | | | |
| P/N value | 7.497 | 8.808 | 0.589 |

As shown in Table 7, the chromogenic time at 37° C. for 15 min was optimal.

### Example 10: Preparation and Use Method of ELISA Detection Method

(1) Coating: The VP6 fusion recombinant protein is diluted to 3.5 μg/mL using a 0.05 mol/L Na₂CO₃—NaHCO₃ buffer solution and 100 μL of the diluted protein solution was added to each well, and adsorbed at 4° C. for 14 h. After the coating was completed, the liquid in the wells was discarded, and 250 μL of PBS wash solution containing 0.05% Tween-20 was added to each well. The wells were washed three times, with the final wash being tapped dry;
(2) Blocking: 200 μL of PBS blocking solution containing 5% BSA and 0.05% Tween-20 is added to each well, and blocked at 37° C. for 1 h. After blocking, the liquid in the wells was discarded, 250 μL of wash solution was added to each well. The wells were washed three times, with the final wash being tapped dry;
(3) Primary antibody incubation: dilute the milk sample to be tested (1:100) or serum sample to be tested (1:200) with sample diluent, 100 μL/well, and incubated at 37° C. for 60 min. The liquid in the wells is discarded, 250 μL of wash solution is added to each well. The wells were washed five times, with the final wash being tapped dry;
(4) Secondary antibody incubation: dilute the enzyme-labeled secondary antibody (1:10000) with antibody diluent, 100 μL/well, incubated at 37° C. for 60 min, discard the liquid in the well, 250 μL of wash solution was added to each well. The wells were washed five times, with the final wash being tapped dry;
(5) Color rendering and reading: 100 μL of a single-component TMB substrate chromogenic solution is added to each well, and the reaction was allowed to proceed in dark at 37° C. for 15 minutes. Subsequently, 100 μL of stop solution is added to each well, and the OD value at 450 nm was read using a microplate reader.

### Example 11: Determination of the Critical Value

The detection method prepared in Example 8 are used to ELISA detection for 24 porcine RV-negative milk and 30 porcine RV-negative serum preserved in the laboratory, and each sample is repeated twice. The OD_{450 nm} reading of each sample is determined and the S/P value is calculated.

The OD_{450 nm} absorbance value of 24 known negative milk is detected, and the mean OD₄₅₀ nm absorbance value of 24 negative milk is 0.154 and the SD value is 0.035, and in order to ensure a 99% confidence interval, the cut off value is the mean value plus 3 times the SD value, and the final cut off value calculated is 0.262.

The OD_{450 nm} absorbance value of 30 known negative serums is detected, and the mean OD_{450 nm} absorbance value of 30 negative serums is 0.271 and the SD value is 0.015, in order to ensure a 99% confidence interval, the cut off value is the mean value plus 3 times the SD value, and the final cut off value calculated is 0.319.

### Example 12: Specific Test

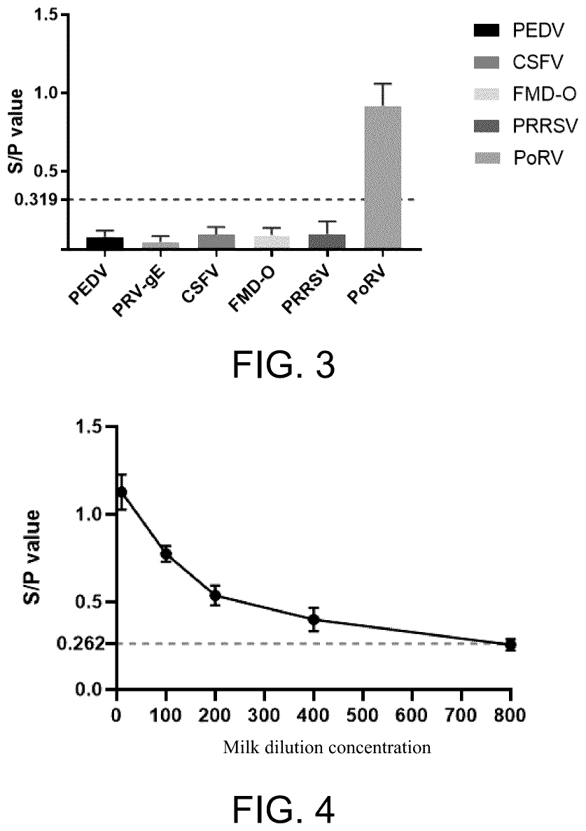

(1) According to the optimized ELSIA method, the standard positive serum of swine fever virus, porcine epidemic diarrhea virus, porcine PRRS virus and foot-and-mouth disease virus (type O) preserved in this laboratory are detected, and PRoV positive and negative serum control is set up to determine whether the PRoV indirect ELISA detection method established in this test had cross-reactivity with antibodies from other major porcine viral pathogens, the results are shown in FIG. 3.

As shown in FIG. 3, the standard positive serum S/P values of swine fever virus, porcine epidemic diarrhea virus, porcine PRRS virus, and foot-and-mouth disease virus (type 0) were all less than 0.319, which shows that PRoV was negative, indicating that the ELISA detection method for VP6 fusion recombinant protein IgA antibody based on against Porcine rotavirus group A established by this invention has good specificity.

### Example 13: Sensitivity Detection

3 PRoV-positive milk were diluted from the ratio of 1:10 and detected using an established indirect ELISA method with a positive and negative control. The sensitivity of this ELISA method is analyzed by measuring the OD_{450 nm} value, and the results are shown in FIG. 4.

The results showed that all 3 positive milk were still positive at a 400 times dilution (OD_{450 nm} value ≥0.262). It was indicated that the ELISA detection method for VP6 fusion recombinant protein IgA antibody based on against Porcine rotavirus group A established by this invention has high sensitivity.

### Example 14: Repeatability Test

(1) Three batches of PRoVVP6 fusion recombinant protein were coated at different times, 3 positive and 3 negative milks were performed by ELISA detection, 4 replicates were set up in the batch, the OD_{450 nm} value is determined, and the mean value, standard deviation and coefficient of variation were calculated to determine the reproducibility within the batchs and the reproducibility between batches, the results are shown in Table 8.

TABLE 8
Repeatability test for ELISA detection method for
VP6 fusion recombinant proteins IgA antibody

| | CV range for 6 milk | median value |
|---|---|---|
| reproducibility within the batches | 7%-16% | 13% |
| reproducibility between batches | 3%-6% | 4% |

As shown in Table 8, the ELISA detection method for VP6 fusion recombinant protein IgA antibody based on against Porcine rotavirus group A variant strains established by this invention has good repeatability.

What is claimed is:

1. An ELISA detection kit for IgA antibody against porcine rotavirus group A, comprising an enzyme-labeled plate coated with a VP6 fusion recombinant protein, a sample diluent, a wash solution, a substrate chromogenic solution, and a stop solution.

2. The detection kit according to claim 1, wherein the VP6 fusion recombinant protein being synthesized use against Porcine rotavirus group A as a template.

3. The detection kit according to claim 1, wherein an encoding sequence of the VP6 fusion recombinant protein is as shown in SEQ ID NO. 1.

4. The detection kit according to claim 1, wherein a coating concentration of the VP6 fusion recombinant protein is 3.5 μg/mL.

5. The detection kit according to claim 1, wherein a coating time of the VP6 fusion recombinant protein is 10-14 h.

6. An ELISA detection method for IgA antibody against Porcine rotavirus group A, comprising the following steps of:
coating: diluting a VP6 fusion recombinant protein to 3.5 μg/mL using a 0.05 mol/L Na₂CO₃—NaHCO₃ buffer solution with a pH of 9.6 to obtained a diluted protein solution, and adding 100 μL of the diluted protein solution to each well such that the VP6 fusion recombinant protein is adsorbed at 4° C. for 14 h;
after the coating, discarding the diluted protein solution in the well, followed by adding 250 L of PBS wash solution containing 0.05% Tween-20 to each of the well, and washing the well for three times, with the final wash being tapped dry;
blocking: adding 200 μL of PBS blocking solution containing 5% BSA and 0.05% Tween-20 to each of the well and blocked at 37° C. for 1 h;
after the blocking, discarding the PBS blocking solution in the well, followed by adding 250 μL of the PBS wash solution to each of the well, and washing the well for three times, with the final wash being tapped dry;
primary antibody incubation: diluting a milk sample to be tested with a sample diluent at a ratio of 1:100 or diluting a serum sample to be tested with the sample diluent at a ratio of 1:200 to obtain a primary antibody solution, adding 100 μL of the primary antibody solution to each of the well and incubated at 37° C. for 60 min;
discarding the primary antibody solution in the well, followed by adding 250 μL of the PBS wash solution to each of the well, and washing the well for five times, with the final wash being tapped dry;
secondary antibody incubation: diluting an enzyme-labeled secondary antibody with an antibody diluent at a ratio of 1:10000 to obtain a secondary antibody solution, followed by adding 100 μL of the secondary antibody solution to each of the well and incubated at 37° C. for 60 min;
discarding the secondary antibody solution in the well, followed by adding 250 μL of the PBS wash solution to each of the well, and washing the well for five times, with the final wash being tapped dry;
chromogenic time and reading: adding 100 μL of a single-component TMB substrate chromogenic solution to each of the well for a chromogenic reaction, and allowing the chromogenic reaction to proceed in dark at 37° C. for 15 minutes, subsequently, adding 100 μL of a stop solution to each of the well, and reading an OD value at 450 nm using an enzyme-labeled instrument;
wherein the method is used for non-disease diagnostic purposes.

7. The method according to claim 6, wherein the VP6 fusion recombinant protein being synthesized use against Porcine rotavirus group A as a template.

8. The method according to claim 6, wherein an encoding sequence of the VP6 fusion recombinant protein is shown in SEQ ID NO.1.

9. The method according to claim 6, wherein a cut-off value of the milk sample to be tested is 0.262;
when the OD value is ≥0.262, a positive test result is obtained, and when the OD value is <0.262, a negative test result is obtained.

10. The method according to claim 6, wherein a cut-off value of the serum sample to be tested is 0.319;
when the OD value is ≥0.319, a positive test result is obtained, and when the OD value is <0.319, a negative test result is obtained.
